(19) United States
(12) Patent Application Publication
(10) Pub. No.: US 2008/0200702 A1
(43) Pub. Date: Aug. 21, 2008

(54) Novel Intermediates, Process for Their Preparation and Process for the Preparation of Coq10 Employing the Said Novel Intermediates

(75) Inventors: Palanivelu Gurumurthy, Chennai (IN); Suneel Manohar Babu Chennamsettyl, Chennai (IN); Vaithyanathan Visweswaran, Chennai (IN)

Correspondence Address: HAMRE, SCHUMANN, MUELLER & LARSON, P.C., P.O. BOX 2902, MINNEAPOLIS, MN, 55402-0902, US

(73) Assignee: NICHOLAS PIRAMAL INDIA LIMITED, Mumbai (IN)

(21) Appl. No.: 11/994,772

(22) PCT Filed: Jun. 21, 2006
(86) PCT No.: PCT/IB2006/052010

§ 371 Date: Apr. 3, 2008

(30) Foreign Application Priority Data
Jul. 6, 2005 (IN) 806/MUM/2005

(51) Int. Cl. C07C 45/81; C07C 43/23; C07C 317/18; C07D 301/02

(52) U.S. Cl. 549/518; 568/362; 568/593; 568/33

(57) ABSTRACT

The present invention relates to an improved process for the preparation of Coenzyme Q. Coenzyme Q₁₀ or CoQ₁₀ has the chemical name 2-[(all-trans)-3,7,11,15,19,23,27,31,35,39-decamethyl-2,6,10,14,18,22,26,30,34,38-tetracontadecaenyl]-5,6-dimethoxy-3-methyl-1,4-benzoquinone and has the formula I.

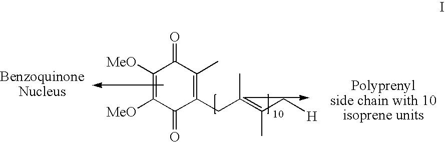

The invention also provides new intermediates useful for the preparation of CoQ₁₀ and processes for their preparation.

### FIELD OF INVENTION

The present invention relates to an improved process for the preparation of Coenzyme Q. Coenzyme Q₁₀ or CoQ₁₀ has the chemical name 2-[(all-trans)-3,7,11,15,19,23,27,31,35,39-decamethyl-2,6,10,14,18,22,26,30,34,38-tetracontadecaenyl]-5,6-dimethoxy-3-methyl-1,4-benzoquinone and has the formula I.

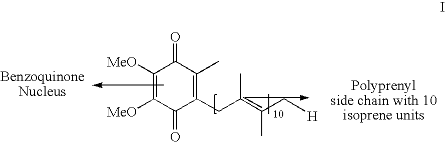

The invention also provides new intermediates useful for the preparation of CoQ₁₀ and processes for their preparation.

### BACKGROUND AND PRIOR ART

This coenzyme is present in virtually in every cell in the human body and is known as the “miracle nutrient”. It plays a vital role in maintaining human health and vigor and is involved in mitochondrial processes such as respiration, maintenance of heart muscle strength, enhancement of the immune system, quenching of free radical in the battle against ageing to name a few (“The miracle nutrient coenzyme” Elsvier/North—Holland Biomedical Press, New York, 1986; “Coenzyme Q: Biochemistry, Bioenergetics, and clinical Applications of Ubiquinone” Wiley, New York, 1985; “Coenzyme Q, Molecular Mechanism in Health and Disease” CRC press).

As depicted above CoQ₁₀ of the formula I comprises mainly of two moieties (i) the head group—“benzoquinone nucleus” and (ii) the “polyprenyl side chain” with ten isoprene units. The source of benzoquinone nucleus is 2,3 dimethoxy 5 methyl benzoquinone, CoQ₀, of the formula 2.

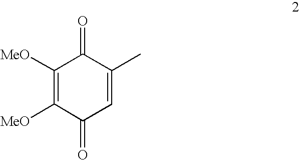

The source of the polyprenyl side chain is solanesol, a naturally occurring alcohol, containing nine isoprene units and having the formula 3.

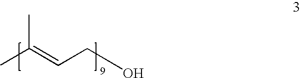

The key step in the synthesis of CoQ₁₀ is in the addition of the remaining isoprene unit.

One of the processes given in literature for the addition of the remaining isoprene unit is by adding the isoprene unit to the “benzoquinone nucleus”. The source of “isoprene unit” is isoprene itself, which is a low boiling liquid of the formula 4.

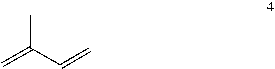

In order to couple the isoprene unit with CoQ₀, of the formula 2, both CoQ₀ and the isoprene units are derivatised to active functional moieties. CoQ₀ is functionalised to bromo derivative with suitable protecting groups to the compound of the formula 5.

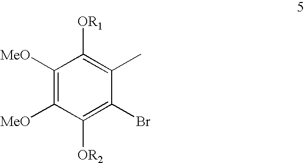

where R₁ and R₂, are protecting groups such as —CH₂ OCH₃, —CH₂ C₆H₅, —CH₃

Isoprene can be functionalised to a) isoprene epoxide of the formula 6 or b) chloroisoprenyl sulphone of the formula 7.

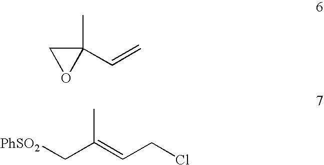

Synthesis of chloroisoprene sulphone from isoprene can lead to positional isomer of the formula 7a and geometrical isomer of the formula 7b.

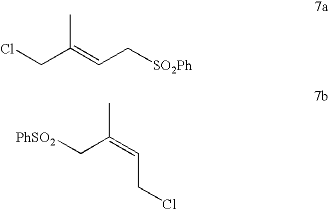

Between isoprene epoxide and chloroisoprenyl sulphone, isoprene epoxide would be a better building block for adding isoprene unit, to the benzoquinone nucleus, as there is no risk of formation of any unwanted isomers.

Literature method of making isoprene epoxide of formula 6 is reported in J. Org. Chem., 25 1673 (1960). The process comprises reacting isoprene with N-bromosuccinimide at a temperature in the range of 18-25° C. for a period in the range of 2-3 hrs. The organic layer formed is extracted with diethyl ether and evaporated to dryness to give crude isoprene bromohydrin. The bromohydrin is added to 30% sodium hydroxide solution and after two hrs the reaction mixture is separated from the aqueous layer. The organic solvent is evaporated to obtain the crude isoprene epoxide, which is purified by atmospheric distillation to obtain the crude of 91% purity in 41% yield.

It is observed that distillation of isoprene epoxide, as described in the above process, leads to polymerization resulting in the formation of undesired compounds and therefore the method is not suitable for industrial scale up.

Isoprene epoxide is attached to the quinone nucleus by condensing with protected functionalised CoQ₀ of formula 5, to form CoQ₁ hydroxy compound of the formula 8 as reported in Sato et al. Chem. Soc. Chem. Commun. (1982) 152.

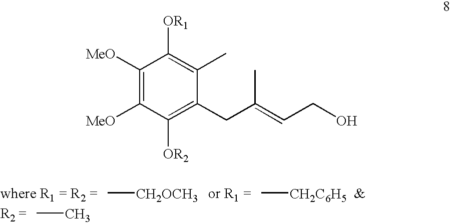

The above method involves coupling of isoprene epoxide to the benzoquinone nucleus by Grignard reaction. Literature does not give any condition of the Grignard reaction. It was observed that formation of Grignard reagent, molar ratio of Grignard reagent to isoprene epoxide, the molar ratio of catalyst and the mode of addition of Grignard reagent and isoprene epoxide, are very critical to the yield and purity of the CoQ₁ of the formula 8. Without these information the process cannot be employed for industrial scale production.

It was observed that the protecting group —CH₂OCH₃ does not withstand the conditions of the Grignard reaction and gets cleaved during the isolation of the product CoQ₁ hydroxy compound of formula 8.

CoQ₁ hydroxy compound of the formula 8 is reacted with n-Butyl Lithium, p-toluene sulphonyl chloride and lithium bromide to give the bromo derivative compound of the formula 9 in 89% yield.

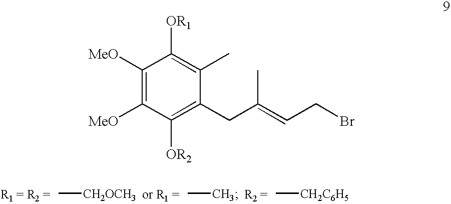

The above method uses expensive reagent like n-butyl lithium and would not be practical for industrial purpose.

The building block of nine isoprene units, the compound of the formula 3, is converted to solanesol sulphone compound of the formula 3a, which is coupled with CoQ₁ bromo compound of the formula 9. This completes the required structure of CoQ₁₀ comprising of “quinone nucleus” and the “polyprenyl chain length of ten isoprene units”, to form the decaprenylated protected CoQ₁₀ sulphone compound of formula 10.

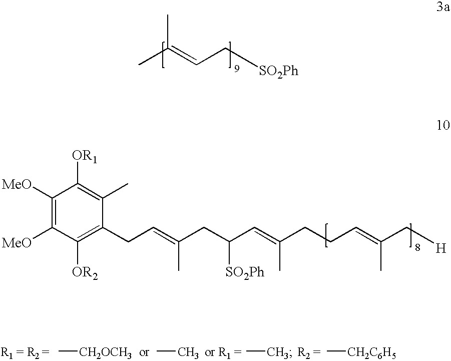

Reaction of solanesol sulphone of the formula 3a with CoQ₁ bromo compound of the formula 9 is reported in Chem. Pharm. Bull 32 3959 (1984), J. Chem. Soc. Chem. Commun. (1982) 153, Chemistry letters 1177 (1986)

The method uses n-butyl lithium in presence of hexamethylphoshphoric triamide (HMPA) in tetrahydrofuran at −70° C. to 0° C. to form the condensed product of the formula 10. n-Butyl lithium and HMPA are costly and hazardous chemicals and are not suitable for large scale manufacture.

The compound of the formula 10 is desulphonated to form the compound of the formula 11a. The desulphonation reaction of 11a gives rise to positional isomers at 5,6 position of the formula 11b. The methods prevalent in the literature for desulphonation are (i) Lithium/ethylamine at −70° C. (ii) modified Bouvault-Blanc method using sodium and ethanol using THF as solvent,

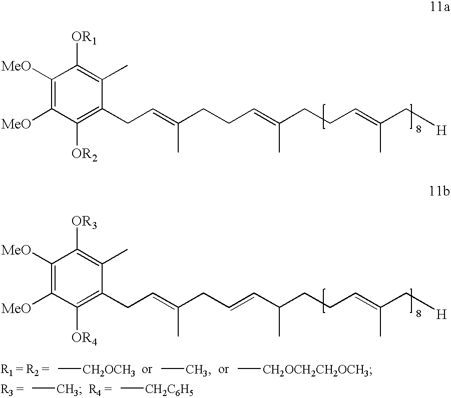

The literature Chem. Pharm. Bull 32 3959 (1984) reports, 69:31 ratio of the desulphonated compounds of the formula 11a, and 11b, which is formed from compound of the formula 10, with methyl protecting groups using the modified Bouvault-Blanc method of sodium and ethanol. The mixture is then purified by silver oxide coated silica gel column chromatography.

Thus using methyl as protected group is not suitable for the industrial manufacture as it gives 31% positional isomer, and also uses expensive method of silver oxide coated silica gel for purification.

Lithium/ethylamine is used when the protecting groups R₁ and R₂ are —CH₂C₆H₅ in the formula 10, that leads to only 7% isomer formation.

Use of lithium/ethylamine leads to the reduction of the aromatic ring and gives rise to impurities. Further the method of Lithium/ethyl amine uses drastic reaction conditions of −70° C. and dry ethylamine. Thus the method of Lithium/ethyl amine for desulphonation is not suitable for the industrial scale manufacture.

Use of —CH₂C₆H₅ as protecting group is not suitable for industrial production as it can be deprotected only by using Lithium/ethyl amine, which as explained above would not be suitable for industrial production.

Literature reports formation of the desulphonated compound of formula 11a with methoxyethoxymethyl as protecting group (R₁ and R₂=methoxyethylmethyl), using “chloro isoprenyl sulphone” compound of formula 7 as the building block of one isoprene unit and “solanesol bromide” compound of formula 3 as building block of nine isoprene units, reported in Bull. Chem. Soc. Japan 55, 1325 (1982). As stated above chloroisoprenyl sulphone gives rise to positional isomers and is not a suitable building block for the industrial synthesis of CoQ₁₀.

The desulphonated compound of the formula 11a is deprotected to form CoQ₁₀ hydroquinone of the formula 12, which is oxidized to form the final CoQ₁₀ Literature method for deprotection uses i) 48% hydrobromic acid at 50° C. (ii) Methanolic Hydrochloric acid Bull. Chem. Soc. Japan 55 1325 (1982).

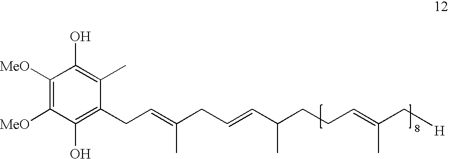

Oxidation of the CoQ₁₀ hydroquinone is carried out by i) aerial oxidation after neutralization of deprotected compound with 10% methanolic potassium hydroxide (ii) silver oxide oxidation and (iii) cerric ammonium nitrate oxidation with methyl protecting groups and (iv) ferric chloride oxidation

It was observed that the coenzymes are sensitive to alkaline medium and neutralization with methanolic potassium hydroxide is not recommended for scale up. The use of aerial oxidation does not take the reaction to completion. Silver oxide and Cerric ammonium nitrate are expensive and therefore their use is not suitable for industrial synthesis of CoQ₁₀.

Ferric chloride is a mild and cheap oxidizing agent, therefore industrially viable.

Use of CoQ₁₀ in broadband medical application is increasing day by day. The key point in the synthesis of CoQ₁₀ is the choice of the “building blocks” of “isoprene unit”, “the benzoquinone nucleus” and “the polyprenyl side chain”. A cost effective process of preparing CoQ₁₀ can be made only with the suitable “building blocks” which are made economically. An industrially viable process is currently lacking.

Keeping the above facts in mind, the inventors explored various alternatives for the preparation of CoQ₁₀, which resulted in developing the following improved novel processes and novel intermediates:

1. Improved processes for the preparation of solanesol bromide and solanesol acetone, the key intermediates for the preparation of the “polyprenyl side chain” of CoQ₁₀. Such processes have been made the subject matter of our copending application no PCT/IB2006/052008
2. Improved process for the preparation of CoQ₁₀, by coupling of the polyprenyl side chain of ten isoprene units, with the head group “bezoquinone nucleus”. Such scheme of synthesis has been made the subject matter of our copending application no PCT/IB2006/052009

The invention disclosed in this application relates to an improved process for the preparation of CoQ₁₀, by condensation of one isoprene unit to the head group “benzoquinone nucleus” to form novel intermediate CoQ₁, which is coupled with solenasyl sulphone.

### Objective of the Invention

The main objective of the present invention is to provide an improved process for the preparation of CoQ₁₀ of the formula I given above overcoming the drawbacks of the hitherto known processes.

Another objective of the present invention is to provide an improved process for the preparation of CoQ₁₀ of the formula I given above which is useful for industrial application

Another objective of the present invention is to provide intermediate, namely, CoQ₁ hydroxy compound of the formula 14, useful in the preparation of coenzymes CoQ₁₀ of formula I

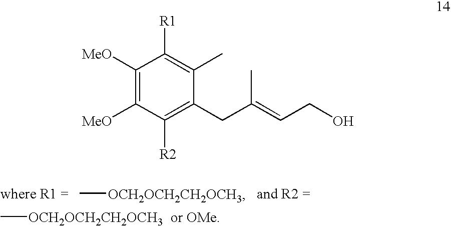

Another objective of the present invention is to provide intermediate, namely, CoQ₁ bromo compound of the formula 15 useful in the preparation of coenzymes CoQ₁₀ of formula I

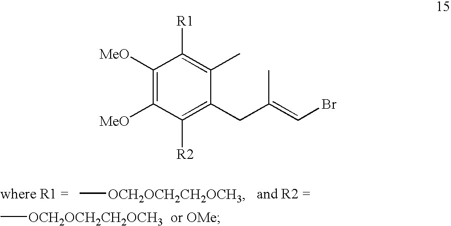

Another objective of the present invention is to provide intermediate namely, CoQ₁₀ Sulphone of the formula 16 useful in the preparation of coenzyme CoQ₁₀ of formula I

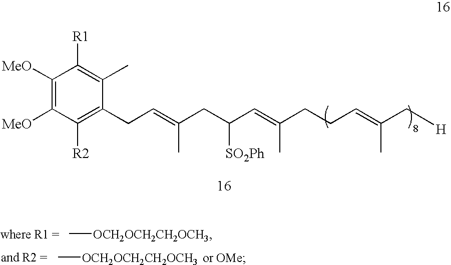

Still another objective of the present invention is to provide an improved process for the preparation of isoprene epoxide of formula 6 which is a key starting material for the process for the preparation of CoQ₁₀ of formula I.

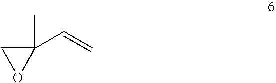

Still another objective of the present invention is to provide an improved process for the preparation of intermediates namely, CoQ₁ hydroxy compounds of the formula 14 wherein the yield is 80% and the purity is 93%, useful for the preparation of CoQ₁₀.

Another objective of the present invention is to provide a process for the preparation of intermediates namely, CoQ₁ bromo compound of the formula 15, which is simple, cost effective and commercially applicable.

Yet another objective of the present invention is to provide a process for the preparation of intermediate namely CoQ₁₀ sulphone of the formula 16, which is simple, cost effective and commercially applicable.

The present inventors have now found that for the preparation of CoQ₁₀ i) isoprene epoxide is a preferred building block for addition of one isoprene unit to CoQ₀ to form intermediates CoQ₁ of the formula 14, ii) solanesol sulphone is a preferred building block with nine isoprene units and iii) the protecting groups to form the building block of benzoquinone nucleus is methoxyethoxy methyl group.

### SUMMARY OF INVENTION

According to an embodiment of the present invention there is provided an improved process for the preparation of coenzyme CoQ₁₀ of formula I,

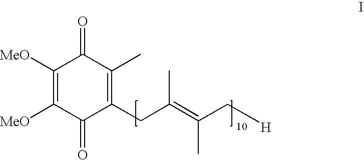

which comprises,
(i) Reacting Grignard reagent of formula 13,

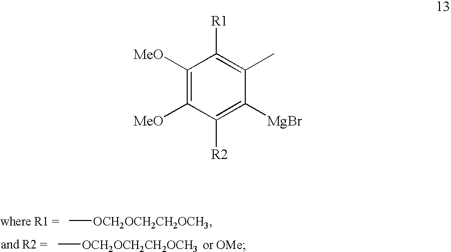

with isoprene epoxide of formula 6

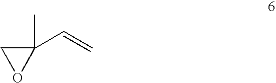

to obtain CoQ₁ hydroxy compound of formula 14,

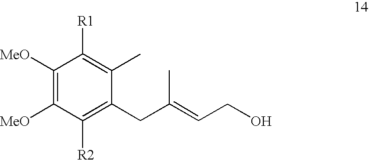

(ii) Brominating by conventional method the compound of formula 14 to obtain a CoQ₁ bromo compound of formula 15,

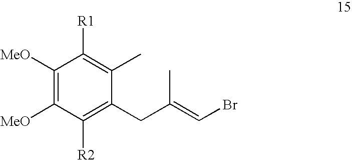

(iii) Condensing by conventional methods, the CoQ₁ bromo compound of formula 15 with solanesol sulphone of formula 3a

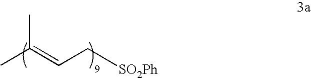

to obtain compound of formula 16,

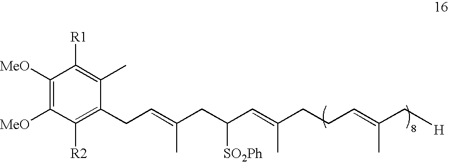

(iv) Desulphonating the compound of formula 16 by conventional method to obtain the compound of formula 11,

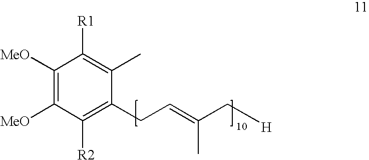

(v) Deprotecting the compound of formula 11 to obtain compounds of formulae 12a or 12b, followed by oxidation to obtain I;

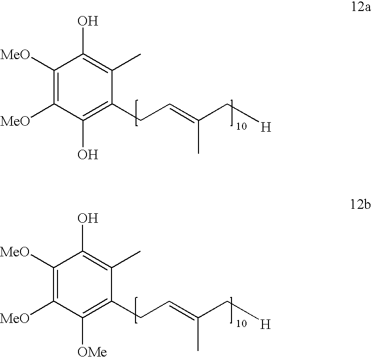

(vi) Crystallizing the crude compound of formula I, and isolating the pure compound of formula I.

According to another embodiment of the present invention, there is provided an improved process for the preparation of CoQ₁₀ of the formula 1,

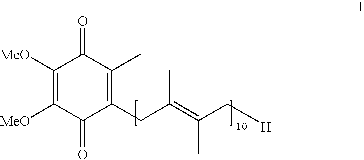

which comprises,
i. Reacting Grignard reagent of formula 13,

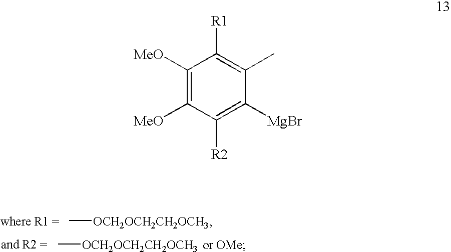

with isoprene epoxide of formula 6 in the presence of copper salt under inert atmosphere, at a temperature in the range of −70° C. to 25° C.;

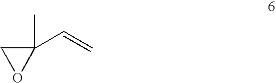

ii. Quenching the resultant reaction mixture formed in step (i) in an acidic or basic medium, extracting with a water immiscible solvent and evaporating to obtain CoQ hydroxy compound of formula 14,

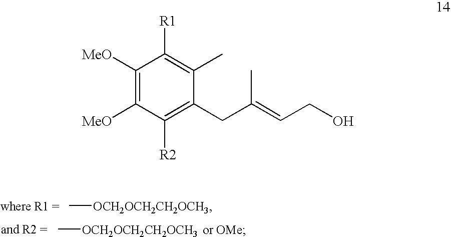

iii. Brominating by conventional methods the compound of formula 14 to obtain a CoQ₁ bromo compound of formula 15, quenching the resultant mixture in an aqueous medium, followed by extracting the compound of formula 15, in a water immiscible solvent, and evaporating the solvent to isolate the compound of formula 15;

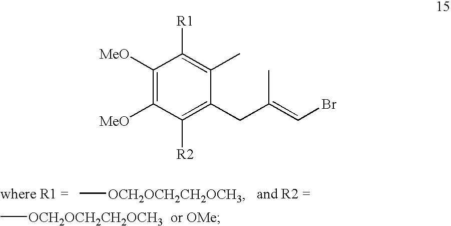

iv. Condensing by conventional methods, the CoQ₁ bromo compound of formula 15 with solanesol sulphone of formula 3a to obtain a compound of formula 16, quenching the resultant reaction mixture with an acidic or basic medium and extracting the resultant compound of formula 16 with a water immiscible solvent, followed by distilling the solvent to isolate the compound of formula 16

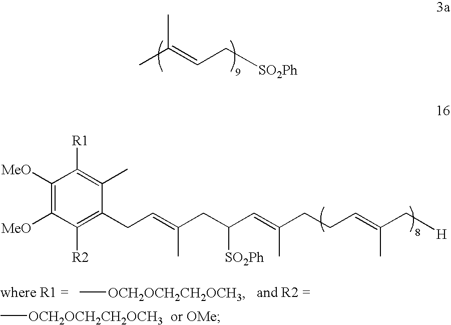

v. Desulphonating the compound of formula 16 by conventional methods to form the compound of formula 11,

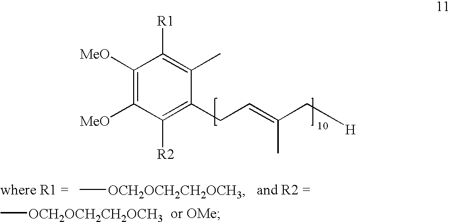

vi. Deprotecting the compound of formula 11 to obtain compounds of formulae 12a or 12b, followed by oxidation by conventional method to obtain I;

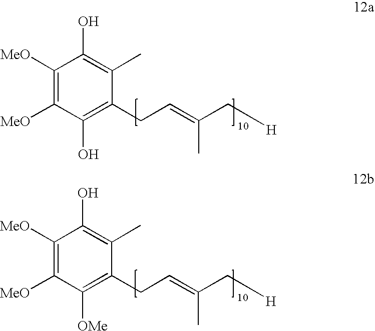

vii. Crystallizing the crude compound of formula I, and isolating the pure compound (about 98%) of formula I.

According to another embodiment of the present invention there is provided an improved process for the preparation of isoprene epoxide of the formula 6, useful in the preparation of coenzyme CoQ₁₀ of formula I

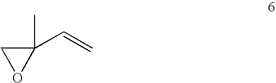

which comprises,
   (i) Treating isoprene of the formula 4 with N-bromosuccinimide at a temperature in the range of 0-25° C. in an aqueous medium;

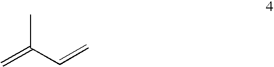

(ii) Extracting the resultant bromohydrin of the formula 6a in a water immiscible organic solvent, followed by distilling the solvent to obtain the crude bromohydrin of the formula 6a;

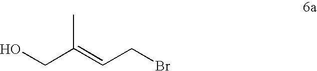

(iii) Distilling the crude bromohydrin of the formula 6a by vacuum distillation to obtain the pure (96%) bromohydrin, adding the pure bromohydrin to alkaline solution at a temperature in the range of 0-25° C. and;
(iv) Separating the organic layer to obtain isoprene epoxide of the formula 6 having 96% purity.

According to another embodiment of the present invention there is provided a process for the preparation of novel CoQ₁ hydroxy compound of the formula 14, useful in the preparation of coenzyme CoQ₁₀ of formula I

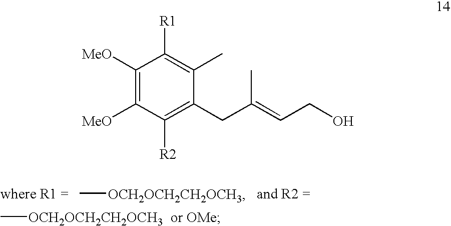

which comprises,
(i) Reacting Grignard reagent of formula 13,

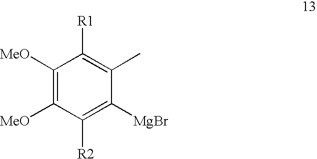

with isoprene epoxide of formula 6 in the presence of copper salt under inert atmosphere, at a temperature in the range of −70° C. to 25° C.,

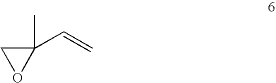

(ii) Quenching the resultant reaction mixture formed in step (i) in an acidic or basic medium, extracting with a water immiscible solvent and evaporating the solvent to obtain CoQ₁ hydroxy compound of formula 14

According to another embodiment of the present invention there is provided a process for the preparation of novel CoQ₁ bromo compound of the formula 15, useful in the preparation of coenzyme CoQ₁₀ of formula I

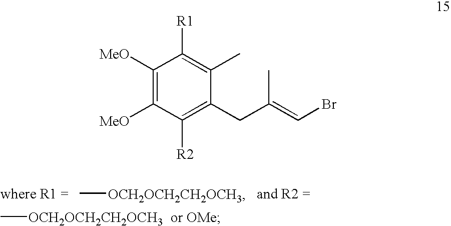

which comprises,
(i) Brominating the hydroxy compound of formula 14 by conventional method,

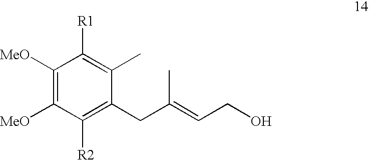

(ii) Quenching the resultant reaction mixture formed in step (i) in an aqueous medium, extracting with a water immiscible solvent and evaporating the solvent to obtain the compound of formula 15.

According to another embodiment of the present invention, there is provided a process for the preparation of the compound of the formula 16, useful in the preparation of coenzyme CoQ₁₀ of formula I

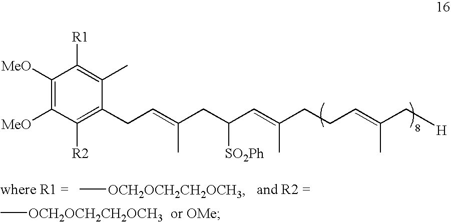

which comprises,
(i) condensation by conventional methods, the intermediate CoQ₁ bromo compound of the formula 15,

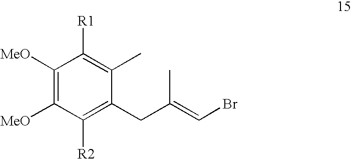

with solanesol sulphone of the formula 3a,

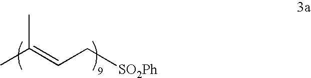

(ii) Quenching the resultant reaction mixture with an acidic or basic medium, extracting with a water immiscible solvent and evaporating the solvent to obtain the compound of the formula 16.

According to another embodiment of the present invention there is provided an improved process for the preparation of the compound of the formula 11, useful in the preparation of coenzyme CoQ₁₀ of formula I

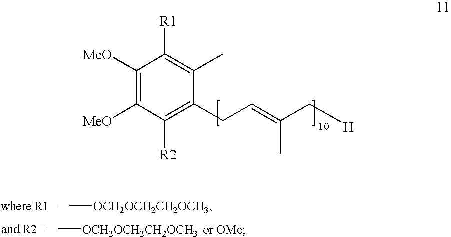

which comprises,
(i) Desulphonating the compound of formula 16

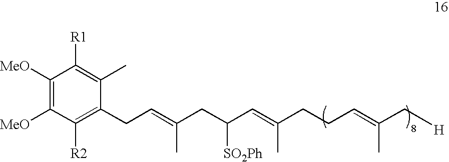

by conventional method, to obtain the compound of the formula 11.

According to another embodiment of the present invention there is provided an improved process for the preparation of the compound of the formula 12a, useful in the preparation of coenzyme CoQ₁₀ of formula I

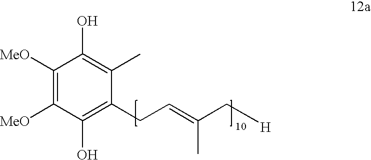

which comprises,
(i) Desulphonating the compound of the formula 16,

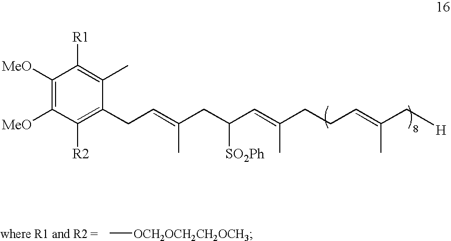

by conventional method, to obtain the compound of formula 11,

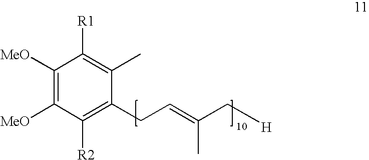

(ii) Deprotecting the resulting compound of formula 11, by conventional method to form the compound of formula 12a

According to another embodiment of the present invention there is provided an improved process for the synthesis of compound of formula 12b, useful in the preparation of coenzyme CoQ₁₀ of formula I

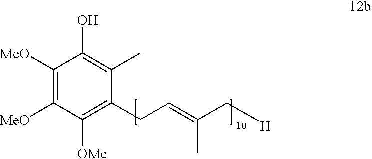

(i) Desulphonating the compound of the formula 16

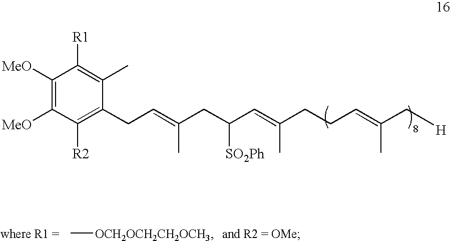

by conventional method, to obtain the novel compound of formula 11,

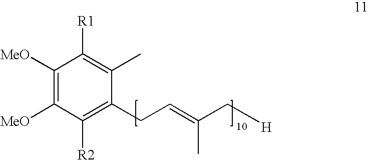

(ii) Deprotecting the compound of the formula 11, by conventional method to form compound of formula 12b.

### DETAILED DESCRIPTION OF THE INVENTION

The present invention provides an improved process for the preparation of the coenzyme CoQ₁₀ of formula 1, as shown in the Scheme-I:

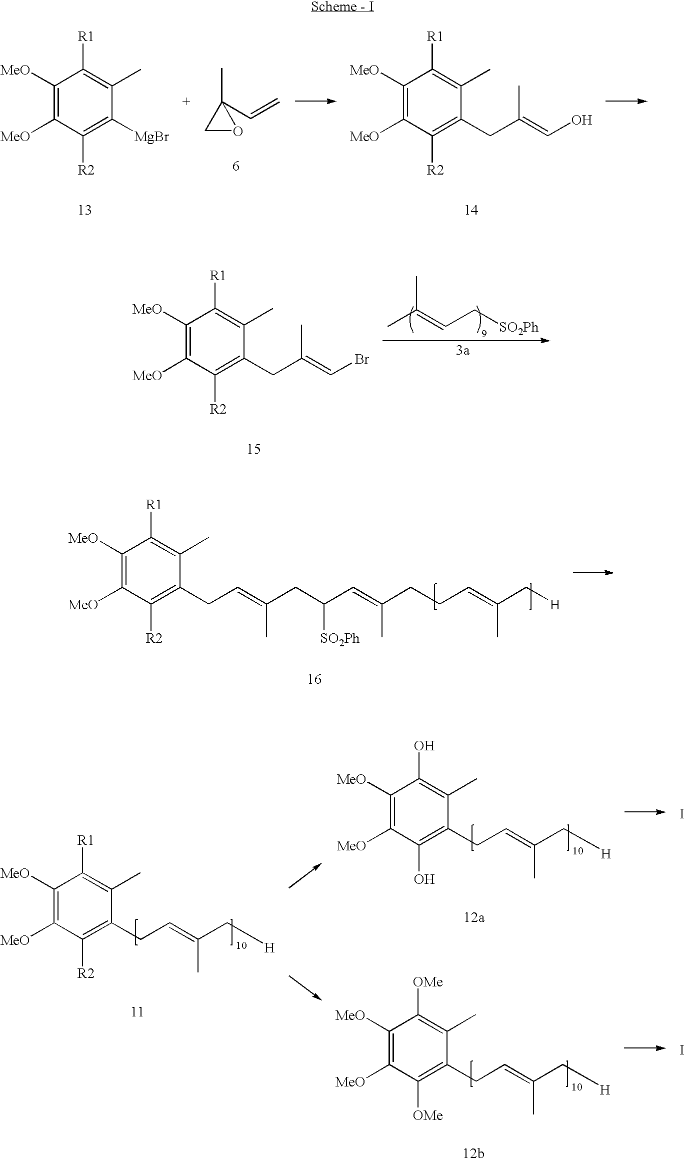

where R1=—OCH₂OCH₂CH₂OCH₃, and R2=—OCH₂OCH₂CH₂OCH₃ or OMe; The process of the present invention which is shown in the Scheme I involves (1) synthesis of building block isoprene epoxide of the formula 6, by an improved method, (2) synthesis of novel CoQ₁ hydroxy compounds of the formulae 14 using methoxyethoxymethyl as protecting groups, (3) synthesis of novel CoQ₁ bromo compounds of the formulae 15 using methoxyethoxymethyl as protecting groups, (4) synthesis of novel decaprenylated CoQ₁₀ sulphone of the formula 16 and (5) desulphonation of the compounds of formulae 16 to form a known compound of the formula 11, (6) by conventional method deprotection of the compound of the formula 11 to form compounds of the formulae 12a or 12b, and (7) oxidation by conventional method of compounds formulae 12a or 12b to form CoQ₁₀ of the formula 1.

Accordingly, the present invention provides an improved process for the preparation of CoQ₁₀ of the formula 1,

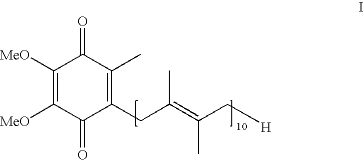

which comprises,
i. Reacting a Grignard reagent of formula 13,

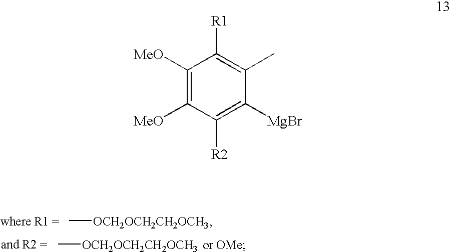

with isoprene epoxide of formula 6 in the presence of copper salt under inert atmosphere, at a temperature in the range of −70° C. to 25° C.;

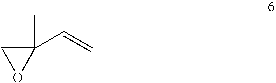

ii. Quenching the resultant reaction mixture formed in step (i) in an acidic or basic medium, extracting with a water immiscible solvent and evaporating the solvent to obtain CoQ₁ hydroxy compound of formula 14,

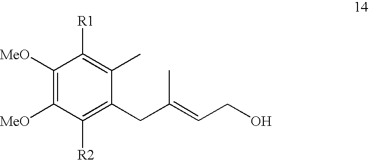

iii. Brominating by conventional method the compound of formula 14 to obtain a CoQ₁ bromo compound of formula 15, quenching the resultant mixture in an aqueous medium, followed by extracting the compound of formula 15, in a water immiscible solvent, and evaporating the solvent to isolate the compound of formula 15;

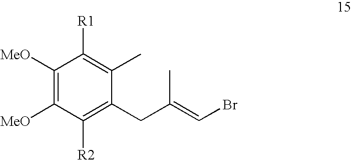

iv. Condensing by conventional methods, the CoQ₁ bromo compound of formula 15 with solanesol sulphone of formula 3a

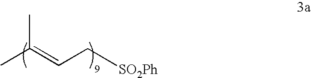

to obtain a compound of formula 16, quenching the resultant reaction mixture with an acidic or basic medium and extracting the resultant compound of formula 16 with a water immiscible solvent, followed by distilling the solvent to isolate the compound of formula 16

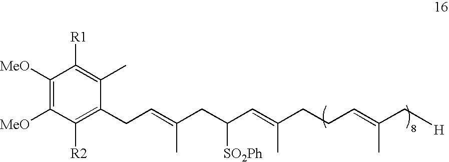

v. Desulphonating the compound of formula 16 by conventional method to form the compound of formula 11,

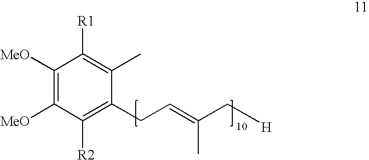

vi. Deprotecting the compound of formula 11 to obtain compounds of formulae 12a or 12b, followed by oxidation by conventional method to obtain I;

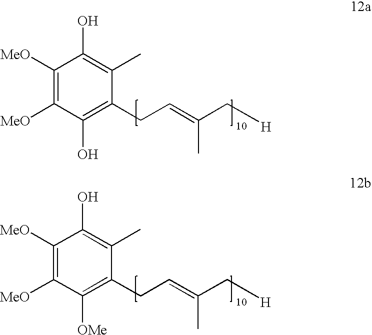

vii. Crystallizing the crude compound of formula I, and isolating the pure compound (about 98%) of formula I.

This method of synthesis of isoprene epoxide by purification at the penultimate step of bromohydrin, makes the process safe and suitable for industrial purpose.

It may be noted that the above description has been given by providing different processes involving preparation of various intermediates, which are known and novel—individually. For a person skilled in the art it would be clear that the process of preparing the CoQ₁₀ according to the improved process disclosed herein, can be conducted continuously starting from solanesol sulphone and appropriate CoQ₁ bromo, without isolation of the various intermediates as illustrated in Schemes I.

According to another embodiment of the present invention there is provided an improved process for the preparation of isoprene epoxide of the formula 6, useful in the preparation of coenzyme CoQ₁₀ of formula I

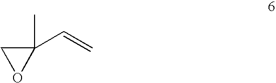

which comprises,
   (i) Treating an isoprene of the formula 4 with N-bromosuccinimide at a temperature in the range of 0-25° C. in an aqueous medium;

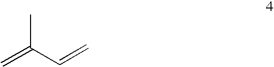

(ii) Extracting the resultant bromohydrin of the formula 6a in a water immiscible organic solvent, followed by distilling the solvent to obtain the crude bromohydrin of the formula 6a;

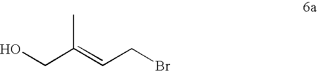

(iii) Distilling the crude bromohydrin of the formula 6a by vacuum distillation to obtain the pure (96%) bromohydrin, adding the pure bromohydrin to alkaline solution at a temperature in the range of 0-25° C. and
(iv) Separating the organic layer to obtain isoprene epoxide of the formula 6 in 96% purity.

According to another embodiment of the present invention there is provided a process for the preparation of novel CoQ₁ hydroxy compound of the formula 14, useful in the preparation of coenzyme CoQ₁₀ of formula I

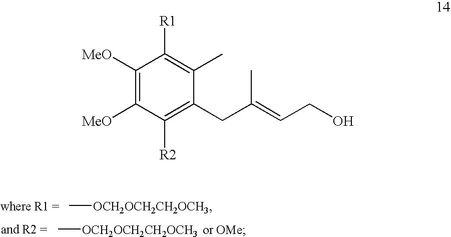

which comprises,
(i) Reacting Grignard reagent of formula 13,

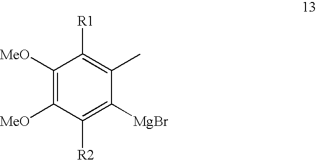

with isoprene epoxide of formula 6 in the presence of copper salt under inert atmosphere, at a temperature in the range of −70° C. to 25° C.,

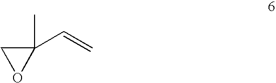

(ii) Quenching the resultant reaction mixture formed in step (i) in an acidic or basic medium extracting with a water immiscible solvent and evaporating the solvent to obtain the compound of formula 14,

According to another embodiment of the present invention there is provided a process for the preparation of novel CoQ₁ bromo compound of the formula 15, useful in the preparation of coenzyme CoQ₁₀) of formula I

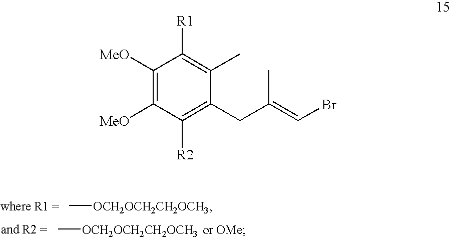

which comprises,
(i) Brominating the hydroxy compound of formula 14 by conventional method,

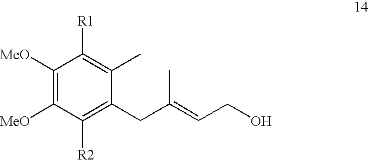

(ii) Quenching the resultant reaction mixture formed in step (i) in an aqueous medium, extracting with a water immiscible solvent and evaporating the solvent to obtain the compound of formula 15.

According to another embodiment of the present invention, there is provided a process for the preparation of the compound of the formula 16, useful in the preparation of coenzyme CoQ₁₀ of formula I

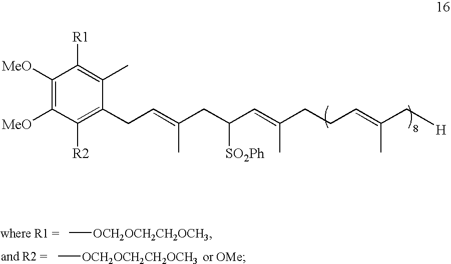

which comprises,
(i) condensation by conventional methods, the intermediate COQ₁ bromo compound of the formula 15,

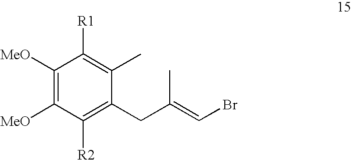

with solanesol sulphone of the formula 3a,

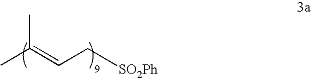

(ii) Quenching the resultant reaction mixture with an acidic or basic medium, extracting with a water immiscible solvent and evaporating the solvent to obtain the compound of the formula 16.

According to another embodiment of the present invention there is provided an improved process for the preparation of the compound of the formula 11, useful in the preparation of coenzyme CoQ₁₀ of formula I

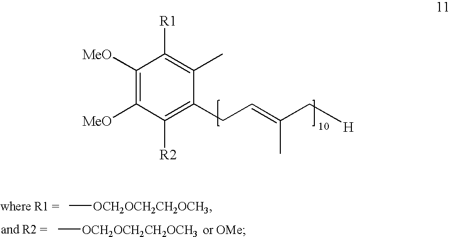

which comprises,
(i) Desulphonating the compound of formula 16

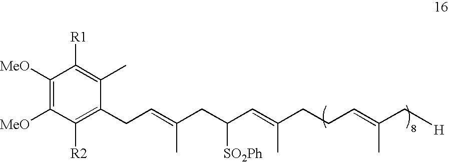

by conventional method, to obtain the compound of the formula 11.

According to another embodiment of the present invention there is provided an improved process for the preparation of the compound of the formula 12a, useful in the preparation of coenzyme CoQ₁₀ of formula I

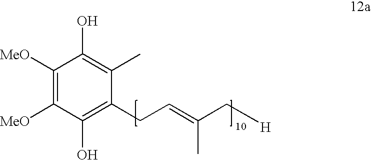

which comprises,
(i) Desulphonating the compound of the formula 16,

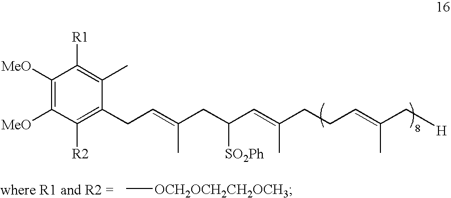

by conventional method, to obtain the compound of formula 11,

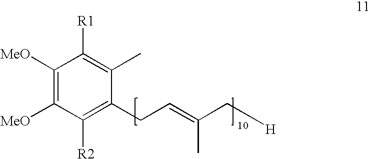

(ii) Deprotecting the resulting compound of formula 11, by conventional method to form the compound of formula 12a

According to another embodiment of the present invention there is provided an improved process for the synthesis of compound of formula 12b, useful in the preparation of coenzyme CoQ₁₀ of formula I

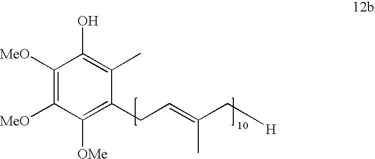

(i) Desulphonating the compound of the formula 16

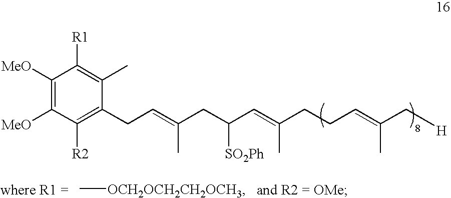

by conventional method, to obtain the novel compound of formula 11,

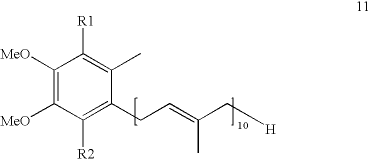

(ii) Deprotecting the compound of the formula 11, by conventional method to form compound of formula 12b.

The details of the various reactions conditions of the processes described above and those preferred ones are given below

The step relating to the preparation of bromohydrin may be carried out by adding N-bromosuccinimide in molar ratio of 1:0.8 to 1:5, preferably 1:1.1. The temperature used may be in the range of 2-25° C., preferably 8-10° C. The reaction mixture may be maintained at 2-25° C., preferably 8-10° C., for 1 to 10 hours preferably 3 hours. The reaction may be worked up by extracting product obtained in a solvent, aromatic or aliphatic hydrocarbon or ether, preferably ether, most preferably diisopropyl ether. The solvent may be distilled to obtain the crude bromohydrin, which may be further distilled to obtain the pure product. The distillation may be carried out at atmospheric pressure or under vacuum 5-30 mm, preferably 8-10 mm. Isoprene epoxide may be synthesized by hydrolyzing the purified bromohydrin obtained as described above in a biphase without employing any solvent. Hydrolysis may be carried out in alkaline medium preferably using sodium hydroxide solution, 5-40% w/v, preferably 30%, at a temperature in then range of 2-25° C. preferably 10-15° C. The separated organic layer of isoprene epoxide may be directly taken for the next step without any further purification.

Preparation of novel intermediate namely, “CoQ₁ hydroxy” compound of the formulae 14 may be carried out by coupling the corresponding Grignard reagent of formula 13 with the isoprene epoxide in the presence of cuprous salt. The Grignard reagent may be prepared by any known method as well by the method described in our co pending application PCT/IB2006/052009.

The coupling reaction may be carried out by treating the appropriate Grignard reagent with cuprous salt like cuprous halide selected from cuprous chloride, cuprous bromide, preferably cuprous chloride or an organic reagent of copper derivative preferably copper acetyl acetone. The mole ratio of cuprous salt to the Grignard reagent used may vary from 1:1 to 1:0.1, preferably 1:0.2. Use of copper catalyst such as copper acetyl acetone is not reported for Grignard coupling of isoprene epoxide and therefore novel. Isoprene epoxide may be dissolved in solvent like ether, or aromatic hydrocarbons preferably ether preferably tetrahydrofuran, and added to the Grignard reagent at a temperature in the range of 0° C. to −70° C., preferably at −50° C. Cuprous salt may also be added to the isoprene epoxide solution. The coupling reaction may then be carried out by adding the Grignard reagent to the isoprene epoxide solution in presence of the copper salt. Preferred mode may be the addition of the isoprene epoxide solution to the Grignard reagent in the presence of copper salt. This mode of reaction allows the Grignard reagent to equilibrate with the cuprous salt to form the copper derivative which would facilitate the coupling with isoprene epoxide. The Grignard reagent may be used in excess or in equivalent ratio or in lesser molar ratio to the isoprene epoxide. In a Grignard reaction the Grignard reagent is always used in excess to the reactant to be coupled. In the present invention isoprene epoxide is used in excess. Isoprene epoxide being a low boiling liquid can be easily removed. Any excess Grignard reagent compound of formula 13, on quenching forms the corresponding aromatic hydrocarbons which are high boiling liquids and can be removed by column chromatography only.

CoQ₁ hydroxy compound of the formula 14 compound may be converted to the corresponding bromo derivatives of the formula 15 by treating it with a brominating agent, preferably phosphorous tribromide in the presence of N,N dimethyl formamide. CoQ₁ hydroxy compound of the formula 14 in N,N dimethyl formamide may be added to the phosphorous tribromide solution in N,N dimethyl formamide at a temperature in the range of 0-25° C., preferably at 10-15° C. Phoshphorous bromide solution in N,N dimethyl formamide may also be added to CoQ₁ hydroxy compound of the formula 14 taken in N,N dimethyl formamide.

This method of conversion of the CoQ₁ hydroxy compound to CoQ₁ bromo compound has to be addressed in a way that the integrity of the double bond be maintained and also the protecting groups remain intact under high acidic condition. In the present invention N,N dimethyl formamide used forms a complex with phosphorous tribromide and allows the reaction to be instantaneous maintaining the integrity of double bond and retaining the protecting groups. Any other solvents like ether, and hydrocarbon do not give the desired compound of required purity.

The condensation of solanesol sulphone with CoQ₁ bromo compound of the formula 15 may be carried out in the presence of a base such as potassium tertiary butoxide. Solanesol sulphone may be prepared by known method. Potassium tertiary butoxide may be added to solanesol sulphone to generate the ion, or to a mixture of solanesol sulphone and the CoQ₁ bromo compound taken together, at a temperature in the range of 0 to −50° C., preferably −20° C. Solvent used may be a mixture of N,N dimethyl formamide, and ether tetrahydrofuran, diisopropyl ether, preferably diisopropyl ether. Use of diisopropyl ether as a water immiscible solvent allows recovery of solvent thereby making the process cost effective and hence commercially viable. Purification at this stage is not needed and proceeded to the next step of desulphonation thereby further making the process not only simple but also cost effective for commercial production.

The desulphonation of the compound of the formula 16 may be carried out by usual procedure employing of Bouevalt Blanc reduction. Sodium and ethanol may be added in lots to the CoQ₁₀ sulphone at a temperature in the range of −40° C. to 20° C. preferably at −20° C.

Deprotection of the compound of the formula 11 to get the respective compound of the formula 12a or 12b may be carried out using conc. HBr in isopropanol warmed to 50° C., or chloroform and zinc bromide or Amberlite-IR 120 in 1-butanol. Deprotection may be carried out in situ without isolating the deprotected compound of formula 12a or 12b.

The Oxidation of the formula 12a or 12b may carried out by known method such as using aerial oxidation, silver oxide, ferric chloride, preferably using Ferric chloride in isopropanol.

Purification of the oxidized product may be carried out with ethanol, ethanol acetone, methanol acetone, isopropanol preferably isopropanol.

The details of the process of the present invention are given in the Examples below which are provided for illustration only and therefore they should not be construed to limit the scope of the invention

### EXAMPLE 1

### Preparation of Bromohydrin ((E)-4-bromo-2-methylbut-2-en-1-ol)

A suspension of Isoprene (200 g) and water (742 ml) was cooled to a temperature in the range of 8-10° C. with vigorous stirring, to which was added N-Bromosuccinimide (521 g) in portions at 8-10° C. The reaction mixture was maintained at 18-22° C. for 2.0 hrs and worked up by extracting in diisopropylether and washing the diisopropylether layer with water followed by saturated sodium chloride solution and dried under sodium sulphate. The diisopropylether layer was distilled under vacuum and the crude bromohydrin (400 g) thus obtained having a G.C purity of 65-75% was subjected to high vacuum distillation at a vapor temperature of 50-54° C. and pressure of 8-10 mm vacuum, Yield of Bromohydrin=208 g (44% of theory) GC=94−96%.

### EXAMPLE 2

### Preparation of Isoprene Epoxide

30% sodium hydroxide solution (336 ml) was cooled to 10° C. and to this was added Bromohydrin ((E)-4-bromo-2-methylbut-2-en-1-ol) (208 g) through a dropping funnel with vigorous stirring at a temperature in the range of 10-15° C. After the addition was over, the reaction mass was maintained at 10° C. for 2.0 hrs and the organic layer was separated, dried over minimum quantity of anhydrous sodium sulphate and decanted to give 96.2 g of isoprene epoxide with purity 95%. Yield=96.2 g (91% of theory) G.C=94-96%.

### EXAMPLE 3

### Preparation of Isoprene Epoxide

Bromohydrin ((E)-4-bromo-2-methylbut-2-en-1-ol) (208 g) was cooled to 10° C. and to this was added 30% sodium hydroxide (336 ml) through a dropping funnel with vigorous stirring at a temperature in the range of 10-15° C. After the addition was over, the reaction mass was maintained at 15° C. for 2.0 hours and the organic layer was separated, dried over minimum quantity of anhydrous sodium sulphate and decanted to give 94.0 g of isoprene epoxide with purity 96%.

### EXAMPLE 4

### Preparation of Isoprene Epoxide

30% potassium hydroxide solution (453 ml) was cooled to 10° C. and to this was added Bromohydrin ((E)-4-bromo-2-methylbut-2-en-1-ol) (208 g) through a dropping funnel with vigorous stirring at a temperature in the range of 10-15° C. After the addition was over, the reaction mass was maintained at 10° C. for 2.0 hours and the organic layer was separated, dried over minimum quantity of anhydrous sodium sulphate and decanted to give 92.5 g of isoprene epoxide with purity 97%, Yield=96.2 g

### EXAMPLE 5

### Preparation of novel 6-(4-hydroxy-3-methyl-2-butenyl)-2,3-dimethoxy-5-methyl hydroquinone 1,4 bis(2-methoxyethoxymethyl)ether (CoQ₁ hydroxy compound)

### (i) Preparation of Grignard reagent 6-Bromo-2,3-dimethoxy-5-methyl-1,4Bis(2-methoxyethoxymethyl ether)

A suspension of magnesium (7.5 g) in tetrahydrofuran (375 ml) was heated to a temperature in the range of 40-45° C. A pinch of iodine and 6-Bromo-2,3-dimethoxy-5-methyl-1,4hydroquinone Bis(2-methoxyethoxymethyl ether) (125 g) was added slowly until initiation of Grignard reaction took place. After completion of addition the reaction was maintained for 2.0 hours at the same temperature to obtain the Grignard reagent 6-Bromo-2,3-dimethoxy-5-methyl-1,4Bis(2-methoxyethoxymethyl ether).

### (ii) Preparation of (CoQ₁ hydroxy compound)

Grignard reagent prepared as in step (i) above was cooled to −50° C. and anhydrous cuprous chloride (5.63 g) was added to it, followed by isoprene epoxide (35.87 g) in THF (65 ml). The reaction was maintained at the same temperature for 3.0 hrs and quenched in saturated ammonium chloride. The product was extracted in ether. The ether layer was washed with water, saturated sodium chloride solution and dried under sodium sulphate. Ether was distilled under vacou at 50° C. to get the novel (CoQ₁ hydroxy compound). Yield=87.5 g (70% of theory) Purity=90% δ(CDCL₃); 1.80 (s, 3H), 2.15 (s, 3H), 3.38 (s, 6H), 3.41 (br,d, 2H), 3.59 (m, 4H), 3.7 (s, 1H), 3.78 (s, 2H), 3.83 (s, 6H), 3.92 (m, 4H), 3.9 (d, 2H), 5.12 (s, 2H), 5.15 (s, 2H), 5.36 (t, 1H).

### EXAMPLE 6

### Preparation of 6-(4-hydroxy-3-methyl-2-butenyl)-2,3-dimethoxy-5-methyl hydroquinone bis(2-methoxyethoxymethyl)ether (CoQ₁ hydroxy compound)

### (i) Preparation of Grignard reagent 6-bromo-2,3-dimethoxy-5-methylhydroquinone bis 12-methoxyethoxymethyl ether

A suspension of magnesium (7.5 g) in tetrahydrofuran (375 ml) was heated to a temperature in the range of 40-45° C. A pinch of iodine and 6-bromo-2,3-dimethoxy-5-methylhydroquinone bis[2-methoxyethoxymethyl ether] (125 g) was added slowly until initiation of Grignard reagent took place. After completion of addition the reaction was maintained for 2.0 hours at the same temperature to get the Grignard reagent of 6-bromo-2,3-dimethoxy-5-methylhydroquinone bis[2-methoxyethoxymethyl ether.

### (ii) Preparation of CoQ₁ hydroxy compound

Grignard reagent obtained by the process described in step (i) above was added through a dropping funnel into isoprene epoxide (35.87 g) dissolved in THF (65 ml) in presence of anhydrous cuprous chloride (5.63 g) at −50° C. The reaction mixture was maintained at −50° C. for 3.0 hrs and quenched in saturated ammonium chloride. The product was extracted in isopropyl ether, the isopropyl ether layer was washed with water, saturated sodium chloride solution and dried under sodium sulphate. The isopropyl ether was distilled under reduced pressure at 50° C. Pale yellow residue was washed with hexane and the hexane layer separated. The residue obtained was dried under high vacuum at 50° C. to obtain CoQ1. Yield=85.6 g δ(CDCL₃); 1.80 (s, 3H), 2.15 (s, 3H), 3.38 (s, 6H), 3.41 (br,d, 2H), 3.59 (m, 4H), 3.7 (s, 1H), 3.78 (s, 2H), 3.83 (s, 6H), 3.92 (m, 4H), 3.9 (d, 2H), 5.12 (s, 2H), 5.15 (s, 2H), 5.36 (t, 1H).

### EXAMPLE 7

### Preparation of 6-(4-hydroxy-3-methyl-2-butenyl)-2,3-dimethoxy-5-methyl hydroquinone bis(2-methoxyethoxymethyl)ether (CoQ₁ hydroxy compound)

### (i) Preparation of Grignard reagent of 6-bromo-2,3-dimethoxy-5-methyl-hydroquinone bis[2-methoxyethoxymethyl ether

A Suspension of magnesium (7.5 g) in tetrahydrofuran (375 ml) was heated to a temperature in the range of 40-45° C. A pinch of iodine and 6-Bromo-2,3-dimethoxy-5-methylhydroquinone bis[2-methoxyethoxymethyl ether] (125 g) was added slowly until initiation of Grignard reagent took place. After completion of the addition the reaction was maintained for 2.0 hrs at the same temperature to get the Grignard reagent of 6-bromo-2,3-dimethoxy-5-methylhydroquinone bis[2-methoxyethoxymethyl ether.

### (ii) Preparation of CoQ₁ hydroxy compound

The reaction mixture obtained in step (i) above was cooled to −50° C. and anhydrous copper acetyl acetone (1.14 g) was added to it, followed by isoprene epoxide (35.87 g) in THF (65 ml). The reaction was maintained at the same temperature for 3.0 hrs and quenched in saturated ammonium chloride. The product was extracted in ether, washed the ether layer with water, saturated sodium chloride solution, dried under sodium sulphate and ether distilled under vacuum at 50° C. to get COQ₁ hydroxy compound, yield 88.9 g

δ(CDCL₃); 1.80 (s, 3H), 2.15 (s, 3H), 3.38 (s, 6H), 3.41 (br,d, 2H), 3.59 (m, 4H), 3.7 (s, 1H), 3.78 (s, 2H), 3.83 (s, 6H), 3.92 (m, 4H), 3.9 (d, 2H), 5.12 (s, 2H), 5.15 (s, 2H), 5.36 (t, 1H)

### EXAMPLE 8

### Preparation of 6-(4-hydroxy-3-methyl-2-butenyl)-2,3,4-trimethoxy-5-methyl hydroquinone methoxyethoxymethyl ether (CoQ₁ hydroxy compound)

### (i) Preparation of Grignard reagent of 6-bromo-1,2,3-dimethoxy-5-methylhydroquinone-2-methoxyethoxymethyl ether

A suspension of magnesium (7.23 g) in tetrahydrofuran (300 ml) was heated to a temperature in the range of 40-45° C. A pinch of iodine and 2,3,4-trimethoxy-5-bromo-6-methylhydroquinone methoxyethoxymethyl ether (100 g) was added slowly until initiation of Grignard reagent took place. After completion of addition the reaction was maintained for 2.0 hrs at the same temperature to get the Grignard reagent of 6-bromo-1,2,3-dimethoxy-5-methylhydroquinone-2-methoxyethoxymethyl ether

### (ii) Preparation of COQ₁ hydroxy compound

The reaction mixture obtained in step (i) above was cooled to −50° C. and anhydrous cuprous chloride (5.4 g) was added, followed by isoprene epoxide (34.5 g) in THF (50 ml). The reaction was maintained at the same temperature for 3.0 hrs and quenched in ammonium chloride. The product was extracted in isopropyl ether. The isopropyl ether layer was washed with water, saturated sodium chloride solution, dried under sodium sulphate and isopropyl ether distilled under vacuum at 50° C. to get COQ₁ hydroxy compound. Yield=87.5 g (70% of theory).

### EXAMPLE 9

### Preparation of 6-(4-bromo-3-methyl-2-butenyl)-2,3-dimethoxy-5-methyl hydroquinone bis(2-methoxyethoxymethyl)ether (CoQ₁ bromo compound)

PBr₃ (37.3 g) was added to DMF (875 ml) at 15° C. and stirred for 1.0 hr, cooled further to a temperature in the range of 5-10° C. and 6-(4-hydroxy-3-methyl-2-butenyl)-2,3-dimethoxy-5-methyl hydroquinone bis(2-methoxyethoxymethyl)ether prepared in Example 5 (87.5 g) in DMF was added drop wise and maintained at the same temperature for 2.0 hrs. The reaction mixture was quenched with water and solid sodium bicarbonate, extracted with ether and the ether layer washed with water, followed by saturated sodium chloride, dried under sodium sulphate and ether distilled under vacuum at 50° C. to obtain pale yellow oil of COQ₁ bromo compound. Yield=85 g (85% of theory) Purity=90%. δ(CDCL₃); 1.80 (s, 3H), 2.13 (s, 3H), 3.39 (s, 6H), 3.41 (br,d, 2H), 3.58 (m, 4H), 3.83 (s, 6H), 3.88 (m, 8H), 5.12 (s, 2H), 5.15 (s, 2H), 5.36 (t, 1H)

### EXAMPLE 10

### Preparation of 6-(4-bromo-3-methyl-2-butenyl)-2,3-dimethoxy-5-methyl bis(2-methoxyethoxymethyl)ether (CoQ₁ bromo compound)

6-(4-hydroxy-3-methyl-2-butenyl)-2,3-dimethoxy-5-methyl bis(2-methoxyethoxy-methyl)ether of Example 5, (87.5 g) was dissolved in DMF and cooled to a temperature in the range of 5-10° C. and PBr₃ (32.0 g) was added via dropping funnel over a period of 1.0-1.5 hours and maintained at a temperature in the range of 5-10° C. for 2.0 hours. After completion of the reaction, water was added followed by sodium bicarbonate. After extraction with isopropyl ether and washing the organic layer with water and brine solution, the isopropyl ether layer was dried with anhydrous sodium sulphate. Isopropyl ether was stripped off at vacuum to give 85 g of a yellow oil of COQ₁ Bromo compound, of purity 90%.

δ(CDCL₃); 1.80 (s, 3H), 2.13 (s, 3H), 3.39 (s, 6H), 3.41 (br,d, 2H), 3.58 (m, 4H), 3.83 (s, 6H), 3.88 (m, 8H), 5.12 (s, 2H), 5.15 (s, 2H), 5.36 (t, 1H).

### EXAMPLE 11

### Preparation of 6-(4-bromo-3-methyl-2-butenyl)-2,3,4-trimethoxy-5-methyl methoxy ethoxymethyl ether (CoQ₁ bromo compound)

PBr₃ (22.1 g) was added to a solution of DMF (500 ml) at 15° C. and stirred for 1.0 hr, cooled further to 5-10° C. and 6-(4-hydroxy-3-methyl-2-butenyl)-2,3,4-trimethoxy-5-methyl methoxyethoxymethyl ether compound formed in example 8 (50.0 g), in DMF was added drop wise and maintained at the same temperature for 2.0 hrs. The reaction mixture was quenched with water and solid sodium bicarbonate was added, followed by extraction with isopropyl ether and washing the isopropyl ether layer with water, followed by sodium chloride, dried under sodium sulphate and isopropyl ether distilled under vacuum at 50° C. to obtain a pale yellow oil of COQ₁ Bromo compound, weight 49.3 g

### EXAMPLE 12

### Preparation of Solanesyl Sulphone

Solanesol (50 g) was dissolved in THF (150 ml) and cooled to a temperature in the range of −10 to −15° C. Phosphorous tribromide (10.8 g) dissolved in THF (25 ml) was added through a dropping funnel and maintained for 2.0 hrs. Solanesyl bromide was precipitated by adding methanol (300 ml) drop wise at the same temperature, filtered, washed with methanol and dried under high vacuum 0.5 mm/30° C. to yield 50 g of solanesyl bromide 98% purity. Solanesyl bromide (50 g) was suspended in DMF (300 ml) and sodium salt of benzene sulfinic acid (14.8 g) was added to it, stirred for 5-6 hrs and precipitated by adding water (180 ml), filtered and slurry washed with methanol dried under vacuum at 30-35° C. for 5.0 hrs to obtain solanesyl sulphone. Yield=50 g (92% of theory) Purity=90%.

### EXAMPLE 13

### Preparation of 6-(5-phenylsulfonyl-3,7,11,15,19,23,27,31,35,39-decamethyltetraconta-,6,10,14,18,22,26,30,34-decaenyl)-2,3-dimethoxy-5-methylhydroquinone bis(2-methoxyethoxymethyl)ether, (CoQ₁₀ Sulphone)

Solanesyl sulphone (116 g) prepared by the process described in Example 12, was dissolved in a mixture of THF (920 ml) and DMF (189 ml) and cooled to −20° C., followed by addition of potassium tertiary butoxide (27.5 g) to generate an anion. 6-(4-bromo-3-methyl-2-butenyl)-2,3-dimethoxy-5-methyl hydroquinone bis(2-methoxyethoxymethyl)ether, prepared by the process described in Example 9 (92.0 g) dissolved in THF (30 ml) was added drop wise to the anion of solanesyl sulphone and maintained for 1.0 hour at −20° C. The temperature of the contents of the flask was raised to room temperature and held for 1.0 hour, the reaction was quenched with ammonium chloride solution and extracted with hexane, washed the hexane layer with water, followed by saturated sodium chloride solution, dried the organic layer under sodium sulphate and distilled under vacuum at 50° C. to obtain a pale yellow viscous oil of (CoQ₁₀ Sulphone). Yield=180 g (99% of theory). Purity=85%.

### EXAMPLE 14

### Preparation of 6-(5-phenylsulfonyl-3,7,11,15,19,23,27,31,35,39-decamethyltetraconta-2,6,10,14,18,22,26,30,34-decaenyl)-2,3-dimethoxy-5-methylhydroquinone his (2-methoxyethoxymethyl)ether (CoQ₁₀ Sulphone)

Solanesyl sulphone prepared by the process described in Example 12 (39.0 g) and 6-(4-bromo-3-methyl-2-butenyl)-2,3-dimethoxy-5-methyl hydroquinone bis(2-methoxy-ethoxymethyl)ether prepared by the process described in Example 9 (30.6 g) were dissolved in THF (307 ml) and (DMF 63 ml) and cooled to a temperature in the range of 0-5° C., followed by addition of potassium tertiary butoxide (8.68 g) in portions. After the completion of addition, the reaction was maintained at a temperature in the range of 0-5° C. for 1.0 hour and then was raised at room temperature to 25° C. and maintained for 1.0 hour. The reaction mixture was quenched the with ammonium chloride solution and extracted with hexane, the hexane layer washed with water, followed by saturated sodium chloride solution, dried the organic layer under sodium sulphate and distilled under vacuum at 50° C. to obtain a pale yellow viscous oil of CoQ₁₀ Sulphone. Yield=60.0 g

### EXAMPLE 15

### Preparation of 6-(5-phenylsulfonyl-3,7,11,15,19,23,27,31,35,39-decamethyltetraconta-2,6,10,14,18,22,26,30,34-decaenyl)-2,3-dimethoxy-5-methylhydroquinone bis(2-methoxyethoxymethyl)ether (CoQ₁₀ Sulphone)

Solanesyl sulphone prepared by the process described in Example 12 (38.0 g) was dissolved in a mixture of isopropyl ether (342 ml) and DMF (38 ml) and cooled to −10° C., followed by addition of potassium tertiary butoxide (9.3 g) in single lot, to generate an anion. 6-(4-Bromo-3-methyl-2-butenyl)-2,3-dimethoxy-5-methyl hydroquinone bis(2-methoxyethoxymethyl)ether prepared by the process described in Example 9, (30.0 g) dissolved in THF (30 ml) was added drop wise to the anion of solanesyl sulphone and maintained for 1.5 hours at −10° C. The temperature of the reaction mass was raised to 25° C. and held for 2.0 hours. The reaction mixture was quenched with ammonium chloride and the isopropyl ether was separated, washed with water, followed by saturated sodium chloride solution, dried under sodium sulphate and distilled under vacuum at 50° C. to obtain a pale yellow viscous oil of CoQ₁₀ Sulphone, Yield=58.0 g

### EXAMPLE 16

### Preparation of 6-(5-phenylsulfonyl-3,7,11,15,19,23,27,31,35,39-decamethyltetraconta-2,6,10,14,18,22,26,30,34-decaenyl)-2,3,4-trimethoxy-5-methylhydroquinone methoxyethoxymethyl ether, (CoQ₁₀ Sulphone)

Solanesyl sulphone prepared by the process described in Example 12 (22.4 g) was dissolved in a mixture of THF (201 ml) and DMF (22.4 ml) and cooled to −20° C., followed by addition of potassium tertiary butoxide (4.9 g) to generate anion of solanesyl sulphone. 6-(4-bromo-3-methyl-2-butenyl)-2,3,4-trimethoxy-5-methyl hydroquinone methoxyethoxymethyl ether Example 11 (15.0 g) dissolved in THF (30 ml) was added drop wise to the anion of solanesyl sulphone and maintained for 1.0 hrs at −20° C. and the temperature of the contents of the flask was raised to room temperature and held for 1.0 hr. The reaction mixture was quenched with ammonium chloride solution and extracted with hexane, the hexane layer was washed with water, followed by saturated sodium chloride solution, dried under sodium sulphate and distilled under vacuum at 50° C. to obtain a pale yellow viscous oil of CoQ₁₀ Sulphone.

### EXAMPLE 17

### Preparation of 6-(3,7,11,15,19,23,27,31,35,39-decamethyltetraconta-2,6,10,14,18,22,26,30,34-decaenyl)-2,3-dimethoxy-5-methylhydroquinone bis(2-methoxyethoxymethyl)ether (CoQ₁₀ hydroquinone)

6-(5-phenylsulfonyl-3,7,11,15,19,23,27,31,35,39-decamethyltetraconta-2,6,10,14,18,22,26,30,34-decaenyl)-2,3-dimethoxy-5-methylhydroquinone bis(2-methoxyethoxymethyl)ether, prepared by the process described in Example 13 (180 g) was dissolved in THF (1080 ml) and cooled to −20° C., followed by addition of ethanol (84.3 g) and sodium (35 g) and maintaining for 10 hrs at the same temperature. The excess sodium was quenched with ethanol, followed by ammonium chloride solution and extracted with hexane, the hexane layer was washed with water followed by saturated sodium chloride, dried under sodium sulphate and distilled under vacuum at 50° C. Crude product was passed through a silica gel column using hexane and ethyl acetate to get a pure product of CoQ₁₀ Hydroquinone. Yield=87 g (55% of theory), Purity=98%

### EXAMPLE 18

### Preparation of 6-(3,7,11,15,19,23,27,31,35,39-decamethyltetraconta-2,6,10,14,18,22,26,30,34-decaenyl)-2,3,4-trimethoxy-5-methylhydroquinone methoxyethoxymethyl ether; (CoQ₁₀ hydroquinone)

6-(5-phenylsulfonyl-3,7,11,15,19,23,27,31,35,39-decamethyltetraconta-2,6,10,14,18,22,26,30,34-decaenyl)-2,3,4-trimethoxy-5-methylhydroquinonemethoxy-ethoxymethyl ether, prepared by the process described in Example 16 (180 g) was dissolved in THF (1080 ml) and cooled to −20° C., followed by addition of ethanol (84.3 g) and sodium (35 g) and maintaining for 10 hrs at the same temperature. The excess sodium was quenched with ethanol, followed by ammonium chloride solution and extracted with hexane, the hexane layer was washed with water followed by saturated sodium chloride, dried under sodium sulphate and distilled under vacuum at 50° C. Crude product was passed through a silica gel column using hexane and ethyl acetate to get a pure product.

### EXAMPLE 19

### Preparation of 6-(3,7,11,15,19,23,27,31,35,39-decamethyltetraconta-2,6,10,14,18,22,26,30,34-decaenyl)-2,3-dimethoxy-5-methyl 1,4-benzhydroquinone (CoQ₁₀)

Purified 6-(3,7,11,15,19,23,27,31,35,39-decamethyltetraconta-2,6,10,14,18,22,26,30,34-decaenyl)-2,3-dimethoxy-5-methylhydroquinone bis(2-methoxy-ethoxymethyl)ether, prepared by the process described in Example 17 (100 g) was dissolved in isopropyl alcohol (2.0 lit), followed by addition of catalytic quantity of conc. HBr. The reaction mixture was warmed to 50° C. and held for 4.0 hrs. The excess HBr was quenched using sodium bicarbonate and filtered through hyflo. To the clear IPA solution containing CoQ₁₀ Hydroquinone, ferric chloride (78.0 g) in water (35 ml) was added, stirred for 3.0 hrs and quenched with water and extracted with hexane. The hexane layer was washed with water, dried under sodium sulphate and distilled under vacuum to obtain a dark red viscous oil which is dissolved in IPA (525 ml) at 50° C. and cooled slowly to 25° C. to get a pale yellow solid which was filtered and washed with sufficient quantity of IPA

### EXAMPLE 20

### Preparation of 6-(3,7,11,15,19,23,27,31,35,39-decamethyltetraconta-2,6,10,14,18,22,26,30,34-decaenyl)-2,3-dimethoxy-5-methyl 1,4-benzhydroquinone (CoQ₁₀)

Purified 6-(3,7,11,15,19,23,27,31,35,39-decamethyltetraconta-2,6,10,14,18,22,26,30,34-decaenyl)-2,3,-dimethoxy-5-methylhydroquinone bis(2-methoxy-ethoxymethyl)ether, prepared by the process described in Example 17 (100 g) was dissolved in chloroform (1.0 lit), followed by addition of zinc bromide and refluxing for 5.0 hrs. After completion of reaction, the reaction mass was cooled and the organic layer was washed with water, the chloroform layer dried over sodium sulphate and distilled under reduced pressure to get yellow viscous oil. Isopropyl alcohol (500 ml) was added and oxidized using ferric chloride (78.0 g) in water (35 ml), stirred for 6.0 hours at a temperature in the range of 40-45° C. and quenched with water and extracted with hexane. The hexane layer was washed with water, dried under sodium sulphate, and distilled under vacuum to obtain dark red viscous oil, which was dissolved in IPA (400 ml) at 50° C. and cooled slowly at 10° C. to get a pale yellow solid which was filtered and washed with sufficient quantity of IPA.

### EXAMPLE 21

### Preparation of 6-(3,7,11,15,19,23,27,31,35,39-decamethyltetraconta-2,6,10,14,18,22,26,30,34-decaenyl)-2,3-dimethoxy-5-methyl 1,4-benzhydroquinone (CoQ₁₀)

Purified 6-(3,7,11,15,19,23,27,31,35,39-decamethyltetraconta-2,6,10,14,18,22,26,30,34-decaenyl)-2,3-dimethoxy-5-methylhydroquinone bis(2-methoxy-ethoxymethyl)ether, prepared by the process described in Example 17 (3.0 g) was dissolved in 1-butanol (60 ml), followed by addition of Amberlite-IR 120 and warmed to temperature in the range of 50-55° C. for 24.0 hours. After completion of reaction, the reaction mass was cooled and resin was recovered. I-Butanol was distilled under reduced pressure completely. To the yellow viscous oil IPA (60 ml) was added and oxidized using ferric chloride (2.34 g) in water (1.15 ml), stirred for 6.0 hrs at a temperature in the range of 40-45° C., quenched with water and extracted with hexane. The hexane layer was washed with water, dried under sodium sulphate, and distilled under vacuum to obtained a dark red viscous oil which was dissolved in IPA (12.0 ml) at 50° C. and cooled slowly to 10° C. to get a pale yellow solid which was filtered and washed with sufficient quantity of IPA.

### EXAMPLE 22

### Preparation of 6-(3,7,11,15,19,23,27,31,35,39-decamethyltetraconta-2,6,10,14,18,22,26,30,34-decaenyl)-2,3,4-trimethoxy-5-methyl-1,4-benzoquinone (CoQ₁₀)

Purified 6-(3,7,11,15,19,23,27,31,35,39-decamethyltetraconta-2,6,10,14,18,22,26,30,34-decaenyl)-2,3,4-trimethoxy-5-methylhydroquinone 1-methoxyethoxymethyl ether, prepared by the process described in Example 17 (100 g) was dissolved in isopropyl alcohol (1.0 lit) followed by addition of catalytic quantity of conc. HBr and warmed to 50° C. and held for 4.0 hrs. The excess HBr was quenched using sodium bicarbonate and filtered through hyflo. To the clear IPA solution containing CoQ₁₀ Hydroquinone, ferric chloride (78.0 g) in water (35 ml) was added, stirred for 3.0 hrs and quenched with water and extracted with hexane. The hexane layer was washed with water, dried under sodium sulphate, and distilled under vacuum to obtain a dark red viscous oil which was dissolved in IPA (525 ml) at 50° C. and cooled slowly to 25° C. to get a pale yellow solid which was filtered and washed with sufficient quantity of IPA, recrystallized from ethanol Yield=41 g, Purity—98%

### Advantages of the Present Invention

1. The process is safe for industrial application as it is simple avoiding chromatography.
2. The route selected for the synthesis of CoQ₁₀ gives rise to novel intermediates of high purity, thereby making the process cost effective.
3. The process results in various novel intermediates, which are, stable and obtained in high yields.
4. The purity of CoQ₁₀ made by the process is very high, not less than 98%.
5. The yield of CoQ₁₀ from solanesol sulphone is also high, namely 50-55%.
6. The process is cost effective and environmentally safe.

1-34. (canceled)

35. A process for the preparation of coenzyme CoQ₁₀ of formula I, 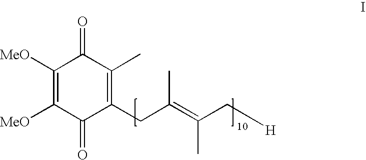
which comprises,
(i) reacting Grignard reagent of formula 13, 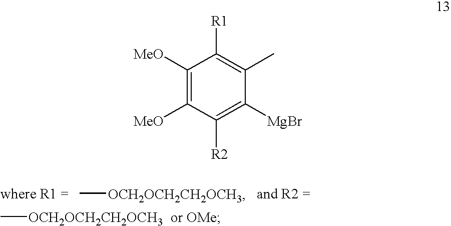
with isoprene epoxide of formula 6 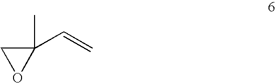
to obtain a CoQ₁ hydroxy compound of formula 14, 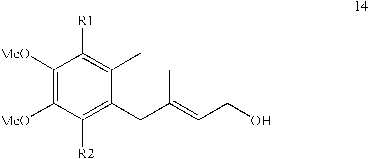
(ii) brominating the compound of formula 14 to obtain a CoQ₁ bromo compound of formula 15, 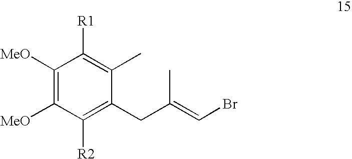
(iii) condensing the CoQ₁ bromo compound of formula 15 with solanesol sulphone of formula 3a 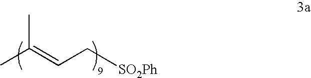
to obtain a compound of formula 16, 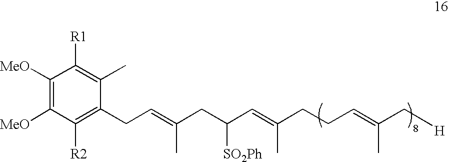
(iv) desulphonating the compound of formula 16 by conventional method to form the compound of formula 11, 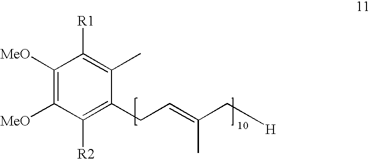
(v) deprotecting the compound of formula 11 to obtain compounds of formulae 12a or 12b, followed by oxidation to obtain I; 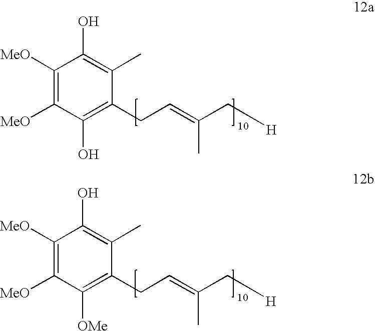
(vi) crystallizing the crude compound of formula I, and isolating the pure compound of formula I.

36. A process for the preparation of coenzyme CoQ₁₀ of formula I as claimed in claim 35, 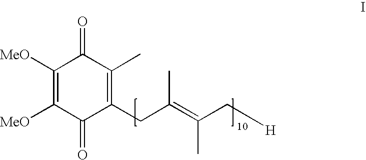
which comprises,
i. reacting Grignard reagent of formula 13, 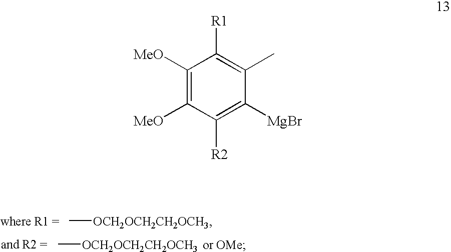
and isoprene epoxide of formula 6 in the presence of copper salt under inert atmosphere, at a temperature in the range of −70° C. to 25° C.; 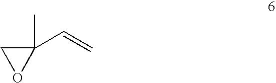
ii. quenching the resultant reaction mixture formed in step (i) in an acidic or basic medium, extracting with a water immiscible solvent and evaporating the solvent to obtain CoQ₁ hydroxy compound of formula 14, 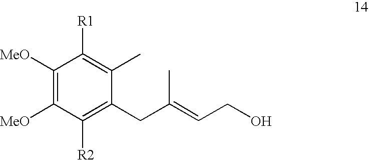
iii. brominating by conventional method the compound of formula 14 to obtain a CoQ₁ bromo compound of formula 15, quenching the resultant mixture in an aqueous medium, followed by extracting the compound of formula 15, in a water immiscible solvent, and evaporating the solvent to isolate the compound of formula 15; 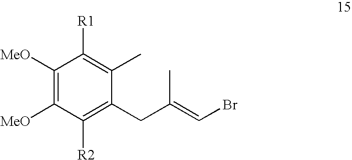
iv. condensing by conventional methods, the CoQ₁ bromo compound of formula 15 with solanesol sulphone of formula 3a 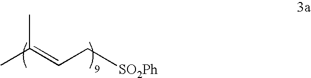
to obtain a compound of formula 16, quenching the resultant reaction mixture with an acidic or basic medium and extracting the resultant compound of formula 16 with a water immiscible solvent, followed by distilling the solvent to isolate the compound of formula 16 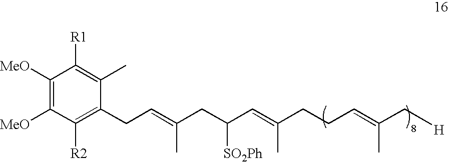
v. desulphonating the compound of formula 16 by conventional method to form the compound of formula 11, 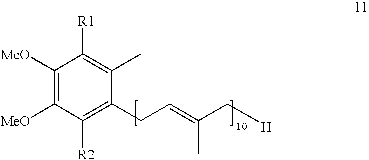
vi. deprotecting the compound of formula 11 to obtain compounds of formulae 12a or 12b, followed by oxidation by conventional method to obtain I; 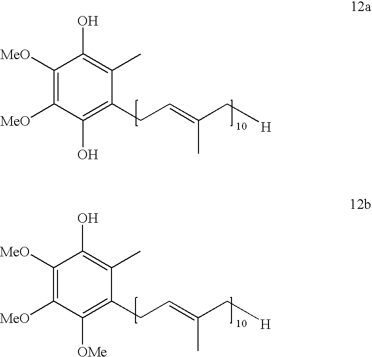
vii. crystallizing the crude compound of formula I, and isolating the pure compound (about 98%) of formula I.

37. A compound of the formula 14,

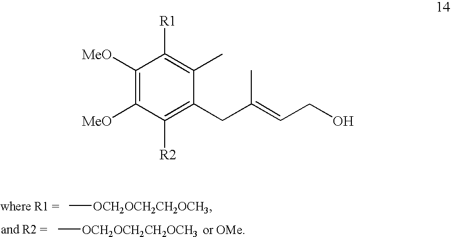

38. A compound of the formula 15,

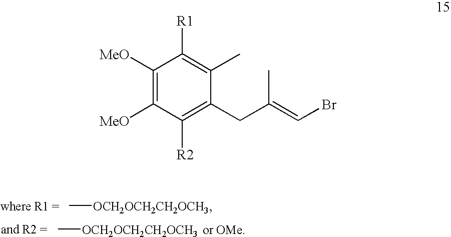

39. A sulphone of the formula 16,

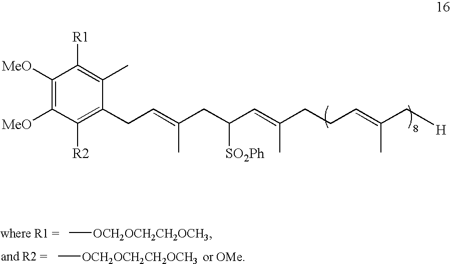

40. A compound of formula Ib,

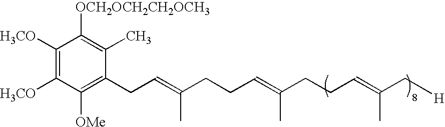

41. A process for the preparation of isoprene epoxide of the formula 6 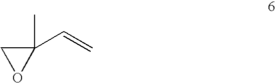
which comprises,
(i) treating an isoprene of the formula 4 with N-bromosuccinimide at a temperature in the range of 0-25° C. in an aqueous medium; 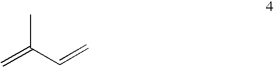
(ii) extracting the resultant bromohydrin of the formula 6a in a water immiscible organic solvent, followed by distilling the solvent to obtain the crude bromohydrin of the formula 6a; 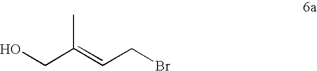
(iii) distilling the crude bromohydrin of the formula 6a by vacuum distillation to obtain the pure (of 96%) bromohydrin, adding the pure bromohydrin to alkaline solution at a temperature in the range of 0-25° C. and;
(iv) separating the organic layer to obtain isoprene epoxide of the formula 6 in 96% purity.

42. A process for the preparation of CoQ₁ hydroxy compound of formula 14, 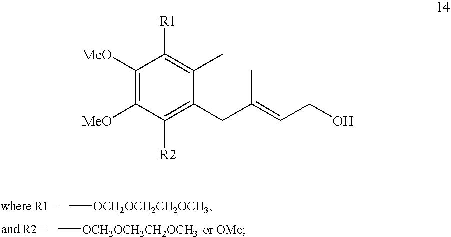
which comprises,
(i) reacting Grignard reagent of formula 13, 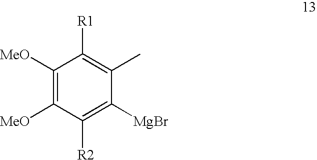
with isoprene epoxide of formula 6 in the presence of copper salt under inert atmosphere, at a temperature in the range of −70° C. to 25° C., 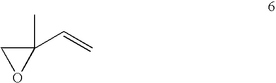
(ii) quenching the resultant reaction mixture formed in step (i) in an acidic or basic medium, extracting with a water immiscible solvent and evaporating the solvent to obtain CoQ₁ hydroxy compound of formula 14.

43. A process for the preparation of CoQ₁ bromo compound of the formula 15, 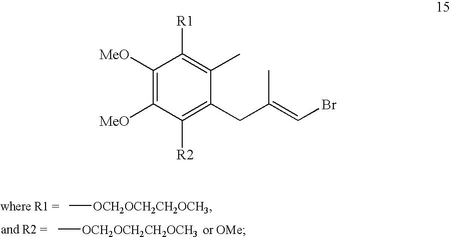
which comprises,
(i) brominating a hydroxy compound of formula 14, 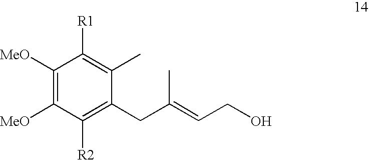
(ii) quenching the resultant reaction mixture formed in step (i) in an aqueous medium, extracting with a water immiscible solvent and evaporating the solvent to obtain the compound of formula 15.

44. A process for the preparation of compound of the formula 16, 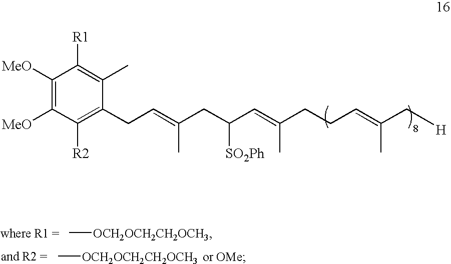
which comprises,
(i) condensing an intermediate COQ₁ bromo compound of the formula 15, 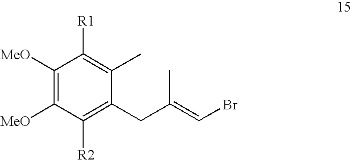
with solanesol sulphone of the formula 3a, 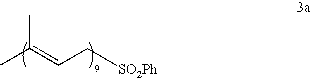
(ii) quenching the resultant reaction mixture with a acidic or basic medium, extracting with a water immiscible solvent and evaporating the solvent to obtain the compound of the formula 16.

45. A process for the preparation of the compound of the formula 11, 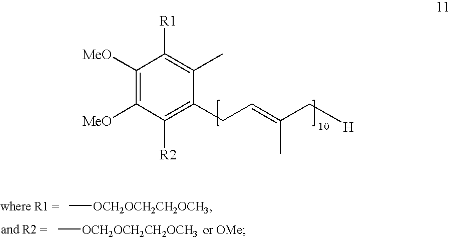
which comprises,
(i) desulphonating a compound of formula 16 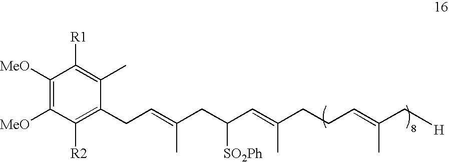
to obtain the compound of the formula 11.

46. A process for the preparation of the compound of the formula 12a, 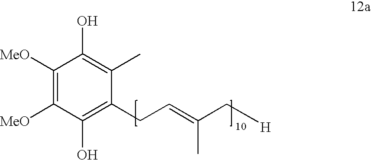
which comprises,
(i) desulphonating a compound of the formula 16, 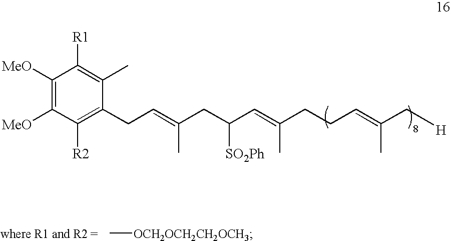
to obtain the compound of formula 11, 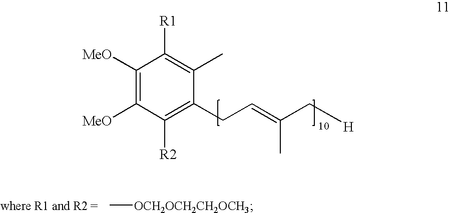
(ii) deprotecting the resulting compound of formula 11, to form the compound of formula 12a.

47. A process for the preparation of compound of formula 12b, 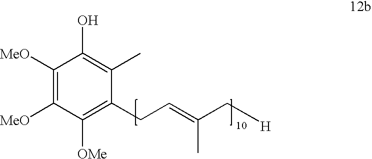
which comprises
(i) desulphonating the compound of the formula 16 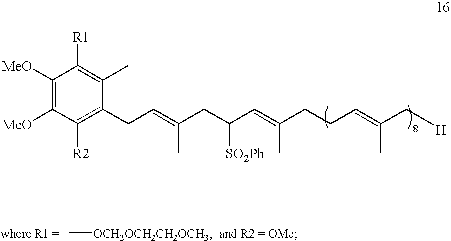
to obtain a compound of formula 11, 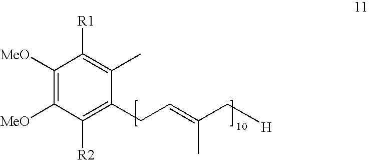
(ii) deprotecting the compound of the formula 11, to form compound of formula 12b.

48. A process as claimed in claim 35 wherein in the step (i) a cuprous salt selected from cuprous halide or an organic reagent of copper derivative is used and the mole ratio of cuprous salt to the Grignard reagent used is in the range from 1:1 to 1:0.1.

49. A process as claimed in claim 36 wherein isoprene epoxide dissolved in solvent is added to the Grignard reagent at a temperature in the range of 0° C. to −70° C., preferably at −50° C.

50. A process as claimed in claim 43 wherein the brominating agent used in step (i) is phosphorous tribromide in the presence of N,N-dimethyl formamide and the bromination is conducted at a temperature in the range of 0-25° C.

51. A process as claimed in claim 41 wherein the preparation of bromohydrin is carried out by adding N-bromosuccinimide in molar ratio of 1:0.8 to 1:5 at a temperature in the range of 2-25° C.

52. A process as claimed in claim 44 wherein the condensation of solanesyl sulphone with CoQ₁ bromo compound of the formula 15 is carried out in the presence of a base selected from potassium tertiary butoxide at a temperature in the range of 0 to −50° C., and the solvent used is selected from the group consisting of N,N dimethyl formamide, ether, tetrahydrofuran and diisopropyl ether.

53. A process as claimed in claim 35, wherein the desulphonation is carried out employing the Bouevalt Blanc reduction.

54. A process as claimed in claim 35, wherein the deprotection of the compound of the formula 11 to get the compound of the formula 12a is carried out using conc. HBr in isopropanol and by warming to a temperature of about 50° C., or chloroform and zinc bromide or Amberlite-IR 120 in 1-butanol, and is carried out in situ without isolating the deprotected compound of formula 12a.

55. A process as claimed in claim 35, wherein the oxidation of the compound formula 12a is carried out by aerial oxidation, using at least one of silver oxide and ferric chloride.

56. A process as claimed in claim 35, wherein crystallisation of the compound of formula I is carried out by using ethanol, ethanol acetone, methanol acetone.

57. A process as claimed in claim 48 wherein the cuprous salt is cuprous chloride or cuprous bromide.

58. A process as claimed in claim 48 wherein the cuprous salt is cuprous chloride.

59. A process as claimed in claim 49 wherein isoprene epoxide is dissolved in ether or aromatic hydrocarbons.

60. A process as claimed in claim 49 wherein isoprene epoxide is dissolved in ether or tetrahydrofuran.

61. A process as claimed in claim 49 wherein isoprene epoxide dissolved in solvent is added to the Grignard reagent at a temperature of −50° C.

62. A process as claimed in claim 50 wherein the bromination is conducted at temperature in the range of 10-15° C.

63. A process as claimed in claim 51 wherein the preparation of bromohydrin is carried out by adding N-bromosuccinimide in molar ratio of 1:1.1.

64. A process as claimed in claim 51 wherein the preparation of bromohydrin is carried out by adding N-bromosuccinimide at a temperature in the range of 8-10° C.

65. A process as claimed in claim 52 wherein the condensation of solanesyl sulphone with CoQ₁ bromo compound of the formula 15 is carried out in the presence of a base selected from potassium tertiary butoxide at a temperature of −20° C.

66. A process as claimed in claim 52 wherein the solvent used is diisopropyl ether.

67. A process as claimed in claim 55, wherein the oxidation of the compound formula 12a is carried out by aerial oxidation, using ferric chloride in isopropanol.

68. A process as claimed in claim 56, wherein crystallisation of the compound of formula I is carried out by using isopropanol.
